(19) United States
(12) Patent Application Publication
(10) Pub. No.: US 2005/0234326 A1
(43) Pub. Date: Oct. 20, 2005

(54) Medical procedure support system and method

(75) Inventors: Akinobu Uchikubo, Iruma-shi (JP); Takeaki Nakamura, Tokyo (JP); Takashi Ozaki, Tokyo (JP); Koichi Tashiro, Sagamihara-shi (JP)

Correspondence Address: Thomas Spinelli;Scully, Scott, Murphy & Presser, 400 Garden City Plaza, Garden City, NY, 11530, US

(73) Assignee: OLYMPUS CORPORATION, TOKYO (JP)

(21) Appl. No.: 11/096,316

(22) Filed: Apr. 1, 2005

(30) Foreign Application Priority Data
Apr. 1, 2004 (JP) 2004-109175

(51) Int. Cl. A61B 5/05

(52) U.S. Cl. 600/407

(57) ABSTRACT

A medical procedure support system includes an image reading unit for reading virtual image data from a storage unit that stores multiple pieces of the virtual image data relating to a subject, which corresponds to steps of a medical procedure, a specifying unit for specifying a step of the medical procedure, and a control unit for controlling the image reading unit based on the step of the medical procedure specified by the specifying unit.

This application claims benefit of Japanese Application No. 2004-109175 filed in Japan on Apr. 1, 2004, the contents of which are incorporated by this reference.

### BACKGROUND OF THE INVENTION

1. Field of the Invention

The present invention relates to a medical procedure support system and method for supporting a medical procedure by creating virtual image data relating to a subject and based on the virtual image data.

2. Description of the Related Art

In recent years, diagnoses using images have been widely performed. Three-dimensional virtual image data of an internal part of a subject is obtained by picking up tomographic images of the subject by, for example, an X-ray CT (Computed Tomography) apparatus. An affected part has been diagnosed by using the virtual image data.

In the beginning of an examination with an X-ray CT apparatus, the X-ray CT apparatus continuously rotates an X-ray irradiating unit with respect to the subject while the subject is being fed continuously in the body axis direction so as to detect an X-ray tomographic image by an X-ray detecting unit. In other words, a helical continuous scan is performed by the X-ray detecting unit in a three-dimensional area of the subject. Furthermore, a signal processing unit of the X-ray CT apparatus creates a three-dimensional image from multiple X-ray tomographic images of continuous slices of the three-dimensional area resulting from continuous scans, which are detected by the X-ray detecting unit.

A three-dimensional image of the bronchi of the lung is one of those three-dimensional images. The three-dimensional image of the bronchi is used for three-dimensionally locating an abnormal part, which may have a lung cancer, for example. In order to check the abnormal part by performing a biopsy, a sample of a tissue thereby is taken by using a biopsy needle or biopsy forceps projecting from a distal part of a bronchi endoscope inserted in the body.

When the abnormal part is close to the end of a branch in a tract in the body having multiple branches like the bronchi, it is hard for the distal end of the endoscope to reach a target part quickly and precisely. Accordingly, Japanese Unexamined Patent Application Publication No. 2000-135215 discloses an apparatus for navigating a bronchi endoscope to a target part.

The navigation apparatus according to Japanese Unexamined Patent Application Publication No. 2000-135215, first of all, creates a three-dimensional tomographic image of a tract in a subject based on image data of a three-dimensional area of the subject. Then, the navigation apparatus obtains a path to a target point along the tract on the three-dimensional tomographic image and creates a virtual path endoscopic image (called virtual image, hereinafter) of the tract along the path based on the image data of the three-dimensional tomographic image. The navigation apparatus then displays the virtual image on a monitor, for example, so as to provide an operator with the virtual image, which is information for navigating a bronchi endoscope to the target part.

Furthermore, conventionally, image analysis software has been in practical use which may be used for a diagnosis of an internal organ of an abdominal area serving as a subject by, in the same manner as above, creating a three-dimensional virtual image of the subject mainly in the abdominal area and displaying the three-dimensional virtual image.

An image system using this kind of image analysis software is used by a doctor for performing a diagnosis for grasping a change in a lesion of a subject in an abdominal area, for example, of a patient in advance before a surgery by viewing a virtual image thereof. The diagnosis with the image system is generally performed outside of an operation room such as a conference room.

### SUMMARY OF THE INVENTION

A medical procedure support system according to a first aspect of the present invention includes: an image reading unit for reading virtual image data from a storage unit that stores multiple pieces of the virtual image data relating to a subject, which correspond to steps of a medical procedure; a specifying unit for specifying a step of the medical procedure; and a control unit for controlling the image reading unit based on the step of the medical procedure specified by the specifying unit.

A medical procedure support system according to a second aspect of the present invention includes: an endoscope having an image pickup unit for picking up an internal part of a body cavity of a subject; an endoscopic image creating unit for creating an endoscopic image from an image signal from the image pickup unit; a storage unit for storing information relating to steps of a medical procedure and virtual image data relating to the subject, which are associated with each other; an image reading unit for reading the virtual image data from the storage unit; a specifying unit for specifying the information relating to a step of the medical procedure under the endoscopic image observation; and a control unit for controlling the reading of virtual image data by the image reading unit based on the information relating to the step of the medical procedure specified by the specifying unit.

A medical procedure support method according to a third aspect of the present invention includes: an image reading step of reading virtual image data from a storage unit that stores multiple pieces of virtual image data relating to a subject, which correspond to steps of a medical procedure; a specifying step of specifying a step of the medical procedure; and a control step of controlling the reading by the image reading step based on the step of the medical procedure specified by the specifying step.

A medical procedure support method according to a fourth aspect of the present invention includes: an endoscopic image creating step of creating an endoscopic image from an image signal from an image pickup unit of an endoscope that picks up an internal part of a body cavity of a subject; a storage step of storing information relating to steps of a medical procedure and virtual image data relating to the subject, which are associated with each other, in a storage unit; an image reading step of reading the virtual image data from the storage unit; a specifying step of specifying the information relating to a step of the medical procedure under the endoscopic image observation; and a control step of controlling the reading of virtual image data by the image reading step based on the information relating to a step of the medical procedure specified by the specifying step.

The other features and advantages of the present invention will be sufficiently apparent from the descriptions below.

### BRIEF DESCRIPTION OF THE DRAWINGS

FIGS. 1 to 19 relate to a first embodiment of the invention;

FIG. 1 is a construction diagram showing a construction of a medical procedure support system;

FIG. 2 is a diagram showing a construction of the endoscope in FIG. 1;

FIG. 3 is a block diagram showing a construction of the main part of the medical procedure support system in FIG. 1;

FIG. 4 is a first diagram for explaining medical procedure virtual images in accordance with the progress of a medical procedure, which are created by the virtual image creating unit in FIG. 1;

FIG. 5 is a second diagram for explaining a medical procedure virtual image in accordance with the progress of a medical procedure, which is created by the virtual image creating unit in FIG. 1;

FIG. 6 is a third diagram for explaining a medical procedure virtual image in accordance with the progress of a medical procedure, which is created by the virtual image creating unit in FIG. 1;

FIG. 7 is a fourth diagram for explaining a medical procedure virtual image in accordance with the progress of a medical procedure, which is created by the virtual image creating unit in FIG. 1;

FIG. 8 is a fifth diagram for explaining a medical procedure virtual image in accordance with the progress of a medical procedure, which is created by the virtual image creating unit in FIG. 1;

FIG. 9 is a sixth diagram for explaining a medical procedure virtual image in accordance with the progress of a medical procedure, which is created by the virtual image creating unit in FIG. 1;

FIG. 10 is a diagram showing a construction of a database unit for storing the medical procedure virtual images in FIG. 5;

FIG. 11 is a flowchart describing an operation during a medical procedure of the medical procedure support system in FIG. 1;

FIG. 12 is a first diagram for explaining the processing in FIG. 11;

FIG. 13 is a second diagram for explaining the processing in FIG. 11;

FIG. 14 is a third diagram for explaining the processing in FIG. 11;

FIG. 15 is a first diagram for explaining a variation example of the medical procedure virtual image in accordance with the progress of a medical procedure, which is created by the virtual image creating unit in FIG. 1;

FIG. 16 is a second diagram for explaining a variation example of the medical procedure virtual image in accordance with the progress of a medical procedure, which is created by the virtual image creating unit in FIG. 1;

FIG. 17 is a third diagram for explaining a variation example of the medical procedure virtual image in accordance with the progress of a medical procedure, which is created by the virtual image creating unit in FIG. 1;

FIG. 18 is a fourth diagram for explaining a variation example of the medical procedure virtual image in accordance with the progress of a medical procedure, which is created by the virtual image creating unit in FIG. 1;

FIG. 19 is a fifth diagram for explaining a variation example of the medical procedure virtual image in accordance with the progress of a medical procedure, which is created by the virtual image creating unit in FIG. 1.

FIG. 20 is a flowchart describing an operation during a medical procedure of a medical procedure support system according to a second embodiment of the invention.

### DESCRIPTION OF THE PREFERRED EMBODIMENTS

A preferred embodiment of the present invention will be described hereinafter.

### First Embodiment

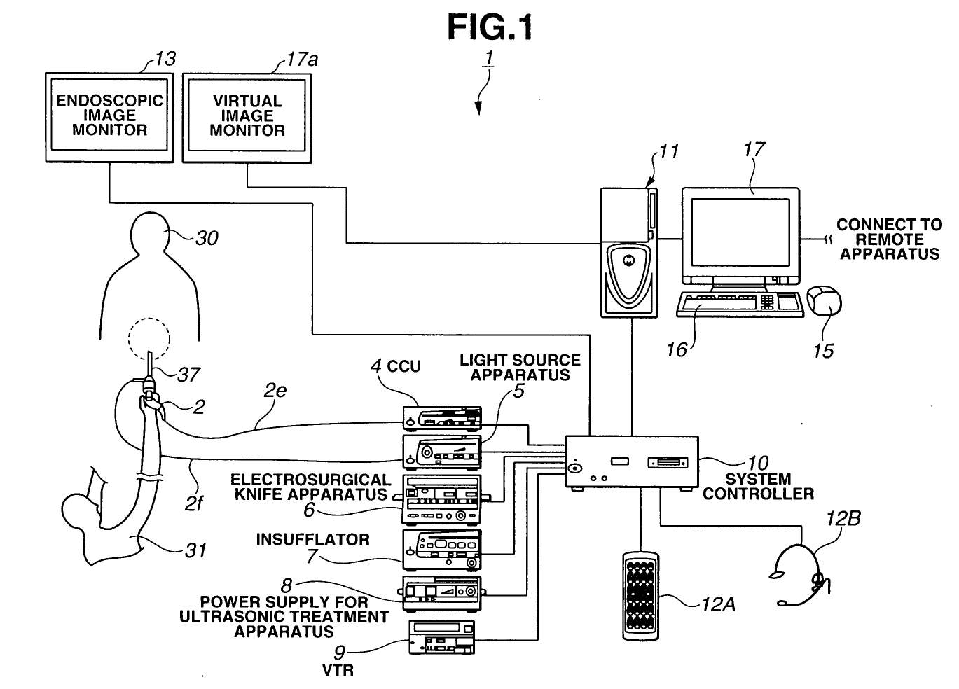

As shown in FIG. 1, a medical procedure support system 1 of the first embodiment is combined with an endoscope system and, more specifically, includes an endoscope 2 serving as an observing unit by which an internal part of the body cavity of a subject can be observed, a CCU 4 serving as an endoscopic image creating device, a light source apparatus 5, an electrosurgical knife apparatus 6, an insufflator 7, a power supply 8 for an ultrasonic treatment apparatus, a VTR 9, a system controller 10, a virtual image creating unit 11, a remote controller 12A, a voice input microphone 12B, a mouse 15, a keyboard 16, a virtual image display monitor 17, and an endoscopic image monitor 13 and virtual image monitor 17a, which are placed in an operation room.

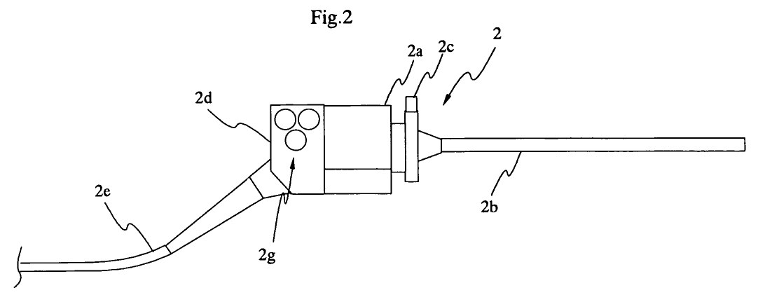

According to the first embodiment, a laparoscope as shown in FIG. 2 is used as the endoscope 2. The endoscope (that is, laparoscope) 2 includes an insertion section 2b to be inserted into the abdominal cavity of the subject and a grip section 2a provided on the proximal side of the insertion section 2b. The endoscope 2 has an illumination optical system and an observation optical system in the insertion section 2b. Thus, an observed part in the abdominal cavity of the subject can be illuminated through the illumination optical system, and an observation image of an internal part of the abdominal cavity of the subject can be obtained by the observation optical system.

The grip section 2a includes a light guide connector 2c connecting to a light guide cable 2f (see FIG. 1). The distal end of the light guide cable 2f is connected to the light source apparatus 5. Under this construction, illumination light from the light source apparatus 5 through the illumination optical system in the insertion section 2b can be irradiated to the observed part.

A camera head 2d having an image pickup unit such as a CCD is connected to an eyepiece, not shown, provided in the grip section 2a, and the camera head 2d includes a remote switch 2g for performing an operation such as zooming-in/-out of the observation image. A camera cable 2e extends on the proximal side of the camera head 2d, and a connector (not shown) is provided at the other end of the camera cable 2e. The connector is used for electrically connecting to the CCU 4.

Referring back to FIG. 1, the insertion section 2b of the endoscope 2 is inserted to a trocar 37 during an operation, and the insertion section 2b is inserted to an abdominal area in the abdomen in the patient with the insertion section 2b held in the trocar 37. The observation image of the abdominal area is obtained by the observation optical system and is formed on the eyepiece. The observation image formed on the eyepiece is picked up by the camera head 2d connecting to the eyepiece, and an image signal output from the camera head 2d is supplied to the CCU 4.

The CCU 4 performs signal processing on the image signal transmitted from the camera head 2d and supplies image data (such as endoscopic live image data) based on the image signal to the system controller 10 placed in an operation room. The system controller 10 selectively outputs image data based on the live still image or moving image from the endoscope 2 from the CCU 4 to the VTR 9. The detail construction of the system controller 10 will be described later.

The VTR 9 can record or play endoscopic live image data from the CCU 4 under the control of the system controller 10. In playing processing, played endoscopic live image data is output to the system controller 10.

The light source apparatus 5 is used for supplying illumination light to a target part of the subject by means of the illumination optical system of the endoscope 2 through the light guide cable 2f.

The electrosurgical knife apparatus 6 is an operation treating apparatus for resecting the abnormal part in the abdominal area of the patient, for example, by using electric heat from an electrosurgical knife probe (not shown). The power supply 8 for an ultrasonic treatment apparatus supplies the power to the operation treating apparatus for resecting or coagulating the abdominal part by using an ultrasonic probe (not shown).

The insufflator 7 includes an air-supply/suction unit, not shown, and supplies carbon dioxide gas to the abdominal area, for example, in the patient through the trocar 37 connected thereto so as to secure a field of view for observation.

The light source apparatus 5, electrosurgical knife apparatus 6, insufflator 7 and power supply 8 for an ultrasonic treatment apparatus are electrically connected to the system controller 10 and are driven under the control of the system controller 10.

The system controller 10, endoscopic image monitor 13 and virtual image monitor 17a are placed in an operation room in addition to equipment such as the CCU 4, VTR 9, light source apparatus 5, electrosurgical knife apparatus 6, insufflator 7 and power supply 8 for an ultrasonic treatment apparatus.

According to the first embodiment, an operator 31 inserts the insertion section 2b to the abdominal part of a patient 30 through the trocar 37 to obtain an image of the subject and performs a treatment on the patient 30 at a position as shown in FIG. 1. In this case, the endoscopic image monitor 13 and virtual image monitor 17a are placed at the positions where the operator 31 looks at easily (in the field-of-view direction).

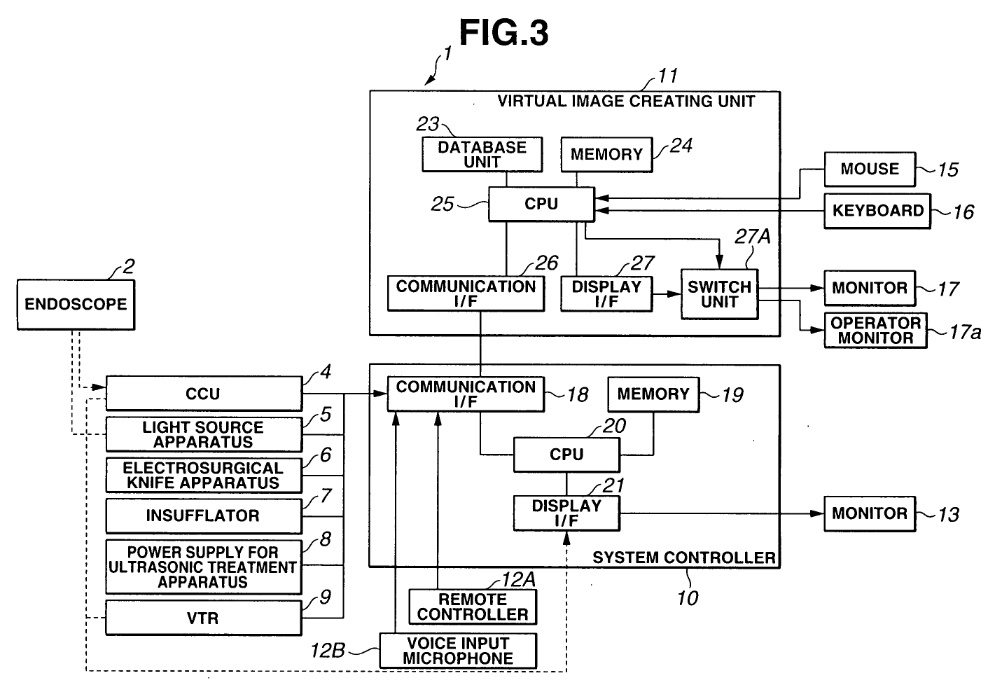

The system controller 10 controls operations (such as display control and light control) of the entire endoscope system. The system controller 10 has, as shown in FIG. 3, a communication interface (called communication I/F, hereinafter) 18, a memory 19, a CPU 20 serving as a controller, and a display interface (called display I/F, hereinafter) 21.

The communication I/F 18 is electrically connected to the CCU 4, light source apparatus 5, electrosurgical knife apparatus 6, insufflator 7, power supply 8 for an ultrasonic treatment apparatus, VTR 9 and virtual image creating unit 11, which will be described later. Transmission and reception of drive control signals or transmission and reception of endoscopic image data in the communication I/F 18 are controlled by the CPU 20. The communication I/F 18 is electrically connected to the remote controller 12A for the operator serving as remote control unit and the voice input microphone 12B serving as a command unit. The communication I/F 18 captures an operation command signal from the remote controller 12A or a voice command signal from the voice input microphone 12B and supplies the operation command signal or voice command signal to the CPU 20.

The remote controller 12A has a white-balance button, an insufflation button, a pressure button, a record button, a freeze button and a release button, a display button, operation buttons, an insertion point button, a focus point button, a display zoom button, a display color button, a tracking button, switch/OK operation button and a numeric keypad.

The white balance button, not shown, is a button for implementing a white balance for an image displayed on the endoscopic image monitor 13 for an endoscopic live image, for example, or the virtual image display monitor 17 or the virtual image monitor 17a.

The insufflation button is a button for starting the insufflator 7 and implementing an insufflation operation.

The pressure button is a button for adjusting to increase or decrease pressure to be used for an insufflation.

The record button is a button for recording the endoscopic live image in the VTR 9.

The freeze button and release button are buttons for commanding to freeze and release during a recording operation.

The display button is a button for displaying the endoscopic live image or virtual image.

The operation buttons include buttons for implementing two-dimensional display (2D-display: display of axial, coronal and sagittal buttons, for example, corresponding to a 2D-display mode) in operation for creating the virtual image.

The insertion point button and focus point button are 3D display operation buttons for implementing three-dimensional display (3D-display) in operation for displaying the virtual image and buttons for selecting the direction of field of view of the virtual image when a 3D display mode is implemented.

More specifically, the insertion point button is a button for displaying information on insertion of the endoscope 2 to the abdominal area, that is, for displaying values in the X, Y and Z directions of the abdominal area to which the endoscope 2 is inserted. The focus point button is a button for displaying a value of the axial direction (angle) of the endoscope 2 in the abdominal area.

The tracking button is used for performing tracking.

The display zoom button is a button for commanding to zoom-in or -out for 3D display and includes a zoom-out button for zooming out a display and a zoom-in button for zooming in a display.

The display color button is a button for changing a display color of a 3D display.

The switch/OK operation button is a button for switching and confirming, for example, setting input information for the operation setting mode confirmed by pressing a button.

The numeric keypad is a button for inputting a numeric value, for example.

Thus, the operator can use the remote controller 12A including these buttons (or a switch) to operate to obtain desired information quickly.

The memory 19 stores image data of endoscopic still images, for example, and data such as equipment setting information, and the data can be stored and read under the control of the CPU 20.

The display I/F 21 is electrically connected to the CCU 4, VTR 9 and endoscopic image monitor 13. The display I/F 21 receives endoscopic live image data from the CCU 4 or endoscopic image data played by the VTR 9 and outputs the received endoscopic live image data, for example, to the endoscopic image monitor 13. Thus, the endoscopic image monitor 13 displays the endoscopic live image based on the supplied endoscopic live image data.

The endoscopic image monitor 13 can also display an equipment setting of the endoscope system and/or setting information such as a parameter in addition to the display of the endoscopic live image under the display control of the CPU 20.

The CPU 20 performs various operations in the system controller 10, that is, the transmission and reception control of various signals via the communication I/F 18 and display I/F 21, writing/reading control of image data to/from the memory 19, display control by the endoscopic image monitor 13 and various operation control based on an operation signal from the remote controller 12A (or a switch).

On the other hand, the virtual image creating unit 11 is electrically connected to the system controller 10.

As shown in FIG. 3, the virtual image creating unit 11 has a database unit 23 for storing a CT image and so on as a storage unit, a memory 24, a CPU 25 serving as an image reading unit, a communication I/F 26, a display I/F 27 and a switching unit 27A.

The database unit 23 includes a CT image data capturing unit (not shown) for capturing DICOM data created by a publicly known CT apparatus, not shown, for picking up X-ray tomographic images of a patient through a portable storage medium such as an MO (Magneto Optical disk) device and a DVD (Digital Versatile Disk) device and stores the captured DICOM image data (CT image data). The reading/writing of the DICOM data is controlled by the CPU 25. The database unit 23 also stores the virtual image, which is a rendering image of each biological part created from the CT image data, in addition to CT image data.

The memory 24 stores data such as the DICOM data and virtual image data created by the CPU 25 based on the DICOM data. The control of storing and reading the data in the memory 24 is performed by the CPU 25.

The communication I/F 26 is connected to the communication I/F 18 of the system controller 10 and transmits and receives a control signal required for an operation to be performed by the virtual image creating unit 11 and the system controller 10 in an interlocking manner. The communication I/F 26 is controlled by the CPU 25 so that the control signal from the system controller 10 can be captured into the CPU 25 through the communication I/F 18.

The display I/F 27 outputs a virtual image created under the control of the CPU 25 to the virtual image monitor 17 or 17a through the switching unit 27A. Thus, the virtual image monitor 17 or 17a displays the supplied virtual image. In this case, the switching unit 27A switches the output of a virtual image under the switching control of the CPU 25 so that the virtual image can be output to a specified one of the virtual image monitors 17 and 17a. If switching the display of a virtual image is not required, the switching unit 27A may be omitted, and a same virtual image can be displayed on both of the virtual image monitors 17 and 17a.

The CPU 25 is electrically connected to the mouse 15 and keyboard 16. In the first embodiment of the present invention, the mouse 15 and keyboard 16 are operation units for inputting and/or defining setting information required for executing an operation for displaying a virtual image by the virtual image display apparatus.

The CPU 25 performs various operations in the virtual image creating unit 11, that is, the transmission and reception control of various signals via the communication I/F 26 and display I/F 27, writing/reading control of image data to/from the memory 24, display control by the monitors 17 and 17a, switching control by the switching unit 27A and various operation control based on an operation signal from the mouse 15 and/or keyboard 16.

The first embodiment may be established as a remote operation support system by connecting the virtual image creating unit 11 to a remote virtual image creating unit through a communication unit.

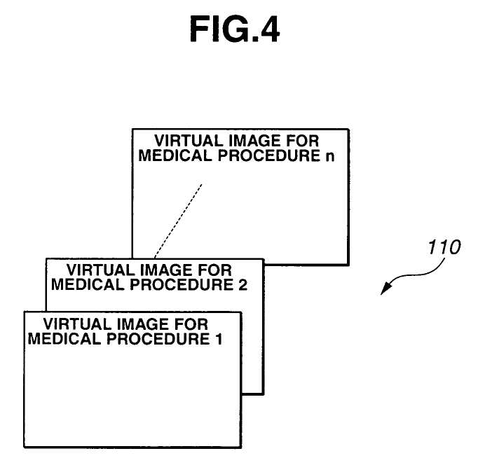

Next, an operation of the first embodiment having the above-described construction will be described. According to the first embodiment, in a case of resecting an organ, for example, the virtual image creating unit 11 creates a medical procedure virtual image 110 in accordance with the progress of a medical procedure as shown in FIG. 4 based on the virtual image of the subject in advance before performing the medical procedure and registers the medical procedure virtual image with the database unit 23.

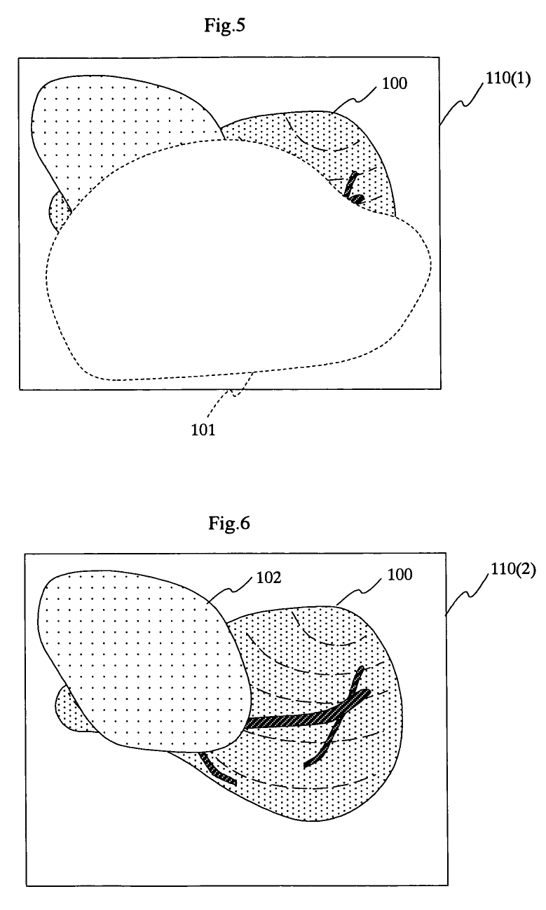

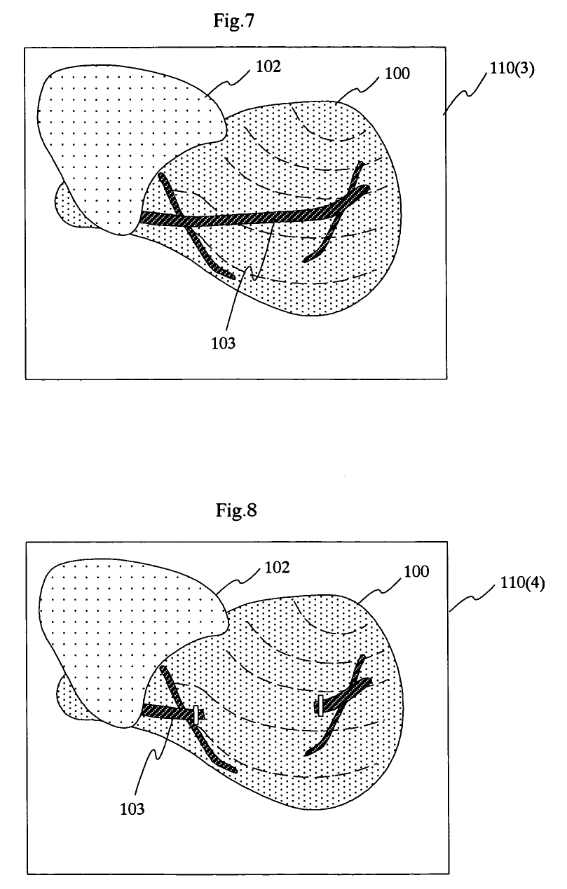

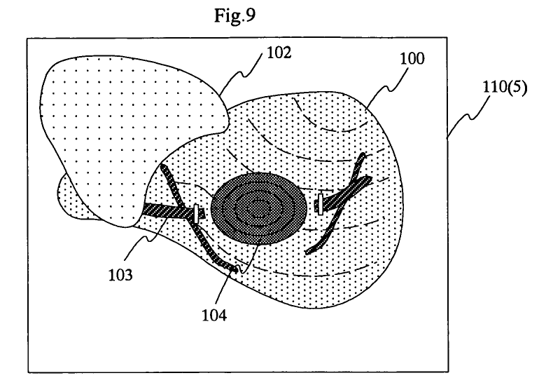

FIGS. 5 to 9 show an example of each medical procedure virtual image. For example, FIG. 5 shows a medical procedure 1 virtual image 110 (1) for checking an arrangement of organs including a focus organ 100 under the observation with the endoscope 2. FIG. 6 shows a medical procedure 2 virtual image 110 (2) resulting from the removal of a fat tissue 101 from the one in FIG. 5. FIG. 7 shows a medical procedure 3 virtual image 110 (3) resulting from moving another organ 102 covering the focus organ 100 in FIG. 6. FIG. 8 shows a medical procedure 4 virtual image 110 (4) in which a blood vessel 103 of the focus organ 100 in FIG. 7 is clipped. FIG. 9 shows a medical procedure 5 virtual image 110 (5) resulting from the removal of an affected tissue 104 of the focus organ 100 from the one in FIG. 8.

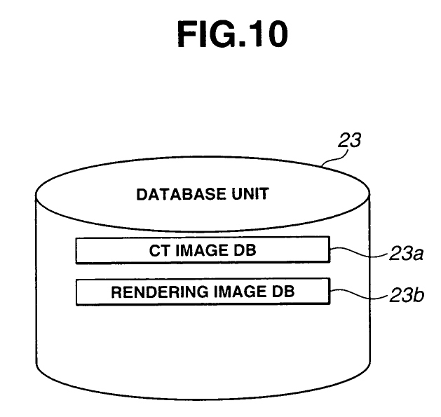

The virtual image creating unit 11 creates a medical procedure virtual image so that a CT image database 23a having DICOM data (CT image data) and a rendering image database 23b having medical procedure virtual images 110 can be established in the database unit 23 as shown in FIG. 10.

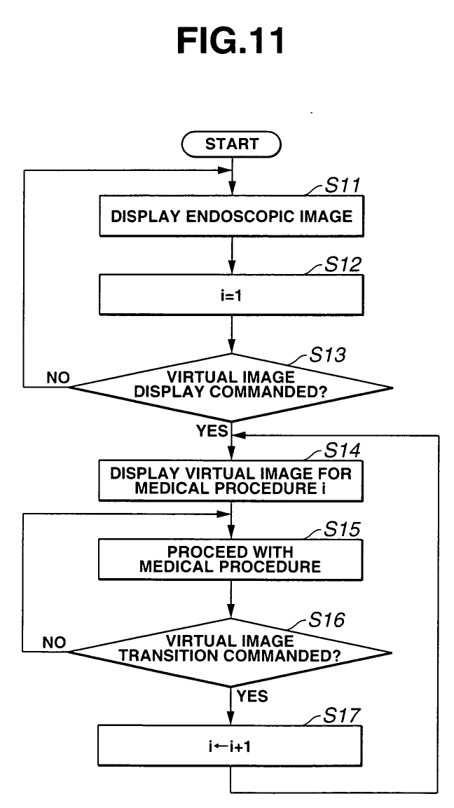

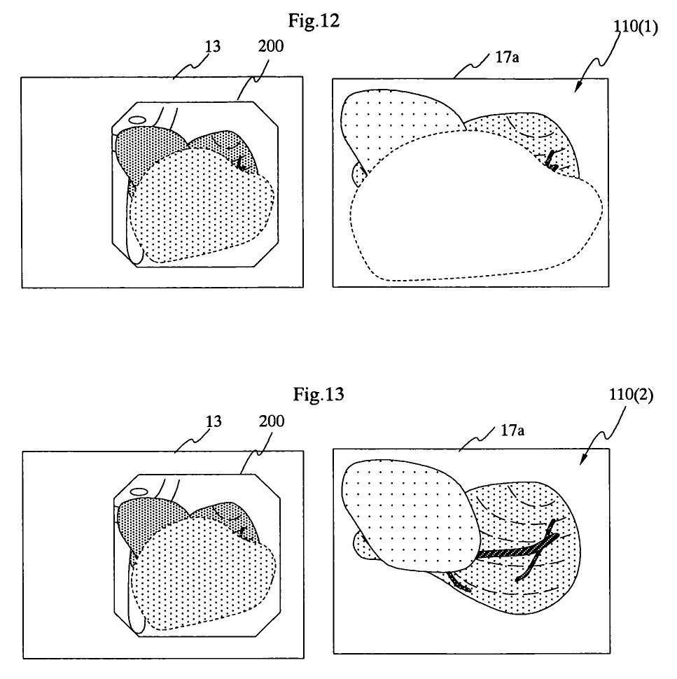

In this way, when the operator starts a medical procedure and the camera head 2d picks up an observation image of an internal part of a subject after the rendering image database 23b is established, an endoscopic image 200 as shown in FIG. 12 is displayed on the endoscopic image monitor 13 in step S11 as shown in FIG. 11. In step S12, one (1) is defined to i.

Then, if the operator produces a voice such as “Virtual display” in accordance with the progress of a medical procedure in step S13, the voice input microphone 12B, for example, detects the voice in step S14, and the system controller 10 recognizes the operator's command by voice recognition processing. Then, if the system controller 10 recognizes the operator's command, the system controller 10 commands the virtual image creating unit 11 to display the medical procedure 1 virtual image 110 (1) shown in FIG. 5 on the monitor 17a, as shown in FIG. 12.

Then, in step S15, a medical procedure is proceeded by referring to the medical procedure 1 virtual image 110 (1) on the monitor 17a and observing the endoscopic image on the monitor 13.

Next, in step S16, if the operator produces a voice such as “Virtually proceed” in accordance with the progress of the medical procedure to command the virtual image transition, the voice input microphone 12B, for example, detects the voice in step S17, and the system controller 10 recognizes the operator's command by voice recognition processing. If the system controller 10 recognizes the operator's command, the system controller 10 increments i and returns to step S14 where the system controller 10 commands the virtual image creating unit 11 to display a medical procedure i virtual image 110 (i) on the monitor 17a as shown in FIG. 13. FIG. 13 shows a state in which the medical procedure 2 virtual image 110(2) shown in FIG. 6 is displayed.

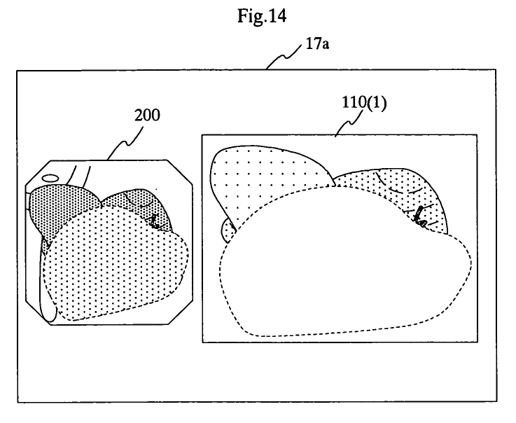

While the endoscopic image is displayed on the monitor 13 and the virtual image is displayed on the monitor 17a here, endoscopic and virtual images may be displayed on the monitor 17a as shown in FIG. 14.

Repeating steps S14 to S17 above results in that the medical procedure virtual images 110 of the medical procedure 1 virtual image 110(1) in FIG. 5→medical procedure 2 virtual image 110(2) in FIG. 6→medical procedure 3 virtual image 110(3) in FIG. 7→medical procedure 4 virtual image 110(4) in FIG. 8→medical procedure 5 virtual image 110(5) in FIG. 9→medical procedure n virtual image 110(n) are sequentially displayed on the monitor 17a based on the operator's voice commands and in accordance with the progress of the medical procedures.

According to the first embodiment, the operator only needs to make a voice command in accordance with the progress (step) of the medical procedure in this way while performing the medical procedure under the observation of endoscopic images on the monitor 13 to display each medical procedure virtual image 110 optimum for reference for the medical procedure. Therefore, the virtual image suitable for medical procedure support can be provided in real time during a medical procedure operation.

Even in a case of performing an operation on the subject in the abdominal area under endoscopic observation, biological image information (such as image information on arrangement of arteries and veins hidden by an organ, for example, and image information on the position of a focus part) on the subject in the observation area of an endoscopic observation image can be provided to the operator more quickly as required.

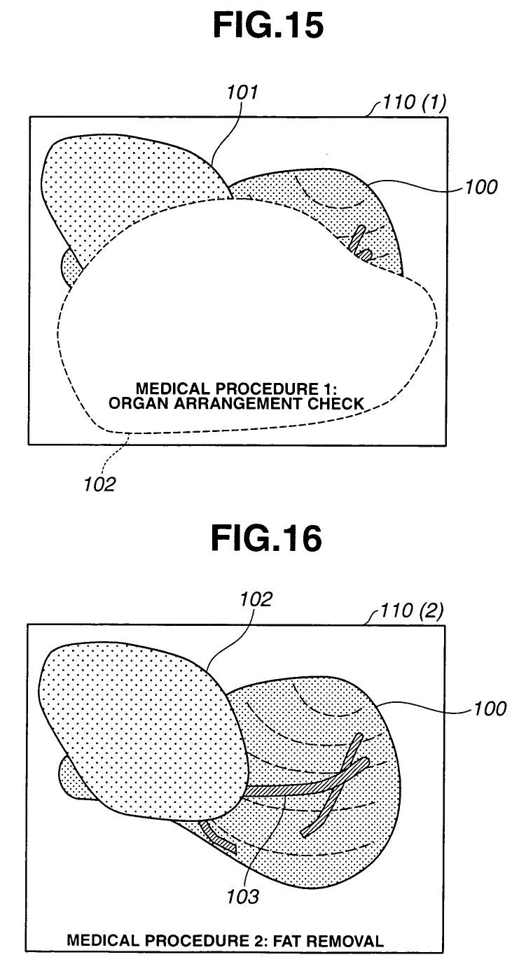

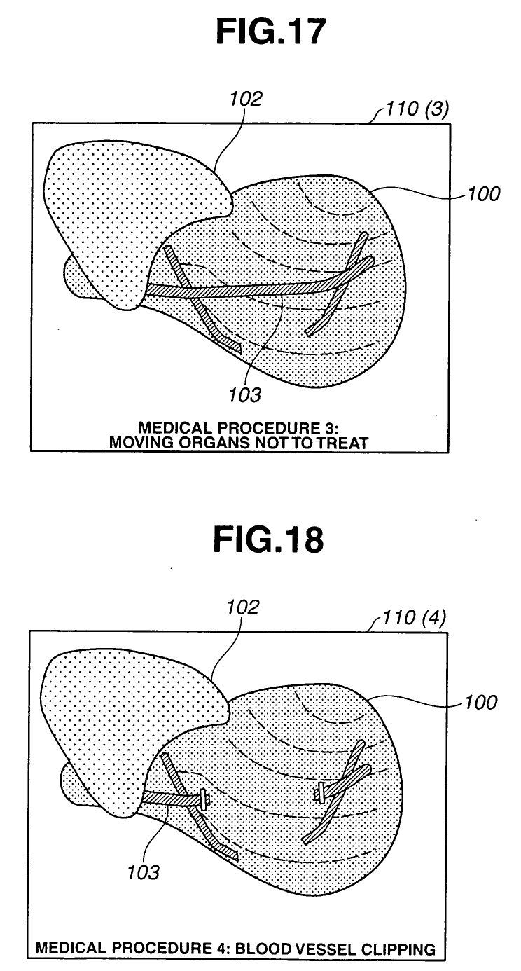

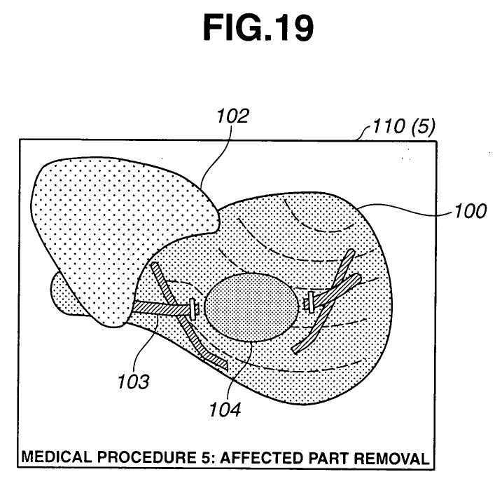

As shown in FIGS. 15 to 19, a content of the medical procedure in accordance with the progress of the medical procedure may be superimposed as text data on each of the medical procedure virtual image 110. FIG. 15 shows a state in which text data, “ORGAN ARRANGEMENT CHECK”, indicating the content of medical procedure 1 is superimposed on the medical procedure 1 virtual image 110(1). FIG. 16 shows a state in which text data, “FAT REMOVAL”, indicating the content of medical procedure 2 is superimposed on the medical procedure 2 virtual image 110(2). FIG. 17 shows a state in which text data, “MOVING ORGANS NOT TO TREAT”, indicating the content of medical procedure 3 is superimposed on the medical procedure 3 virtual image 110(3). FIG. 18 shows a state in which text data, “BLOOD VESSEL CLIPPING”, indicating the content of medical procedure 4 is superimposed on the medical procedure 4 virtual image 110(4). FIG. 19 shows a state in which text data, “AFFECTED PART REMOVAL”, indicating the content of medical procedure 5 is superimposed on the medical procedure 5 virtual image 110(5).

### Second Embodiment

Next, the second embodiment of the present invention, that is only different points from the first embodiment, will be described with reference to FIGS. 1 to 3, and 20.

While the medical procedure virtual image for supporting the medical procedure only in a specific direction of approach can be provided according to the first embodiment, the direction of approach to an affected part of a treatment device such as forceps may be generally determined in the beginning of a medical procedure under endoscopic observation and depending on the position of the affected part in the focus organ.

Accordingly, the second embodiment includes estimating multiple directions of approach to the affected part before the medical procedure, creating the medical procedure virtual image in accordance with the progress of the medical procedure for each estimated direction of approach and storing the result in the rendering image database 23b of the database unit 23. The medical procedure virtual image in accordance with the progress of the medical procedure for each direction of approach stored in the rendering image database 23b is managed by the CPU 20 in a table of approach 1, table of approach 2 and table of approach 3 shown in Tables 1 to 3, for example, for each direction of approach.

TABLE 1
Table of approach 1

| Medical | Names of medical | |
|---|---|---|
| procedure no. | procedures | Names of image files |
| 1 | Organ arrangement | Image 1-1 |
| | check | |
| 2 | Fat removal | Image 1-2 |
| 3 | Moving organs not | Image 1-3 |
| | to treat | |
| 4 | Blood vessel | Image 1-4 |
| | clipping | |
| 5 | Affected part | Image 1-5 |
| | removal | |
| : | : | : |
| : | : | : |

TABLE 2
Table of approach 2

| Medical | Names of medical | |
|---|---|---|
| procedure no. | procedures | Names of image files |
| 1 | Organ arrangement | Image 2-1 |
| | check | |
| 2 | Fat removal | Image 2-2 |
| 3 | Moving organs not | Image 2-3 |
| | to treat | |
| 4 | Blood vessel | Image 2-4 |
| | clipping | |
| 5 | Affected part | Image 2-5 |
| | removal | |
| : | : | : |
| : | : | : |

TABLE 3
Table of approach 3

| Medical | Names of medical | |
|---|---|---|
| procedure no. | procedures | Names of image files |
| 1 | Organ arrangement | Image 3-1 |
| | check | |
| 2 | Fat removal | Image 3-2 |
| 3 | Moving organs not | Image 3-3 |
| | to treat | |
| 4 | Blood vessel | Image 3-4 |
| | clipping | |
| 5 | Affected part | Image 3-5 |
| | removal | |
| : | : | : |
| : | : | : |

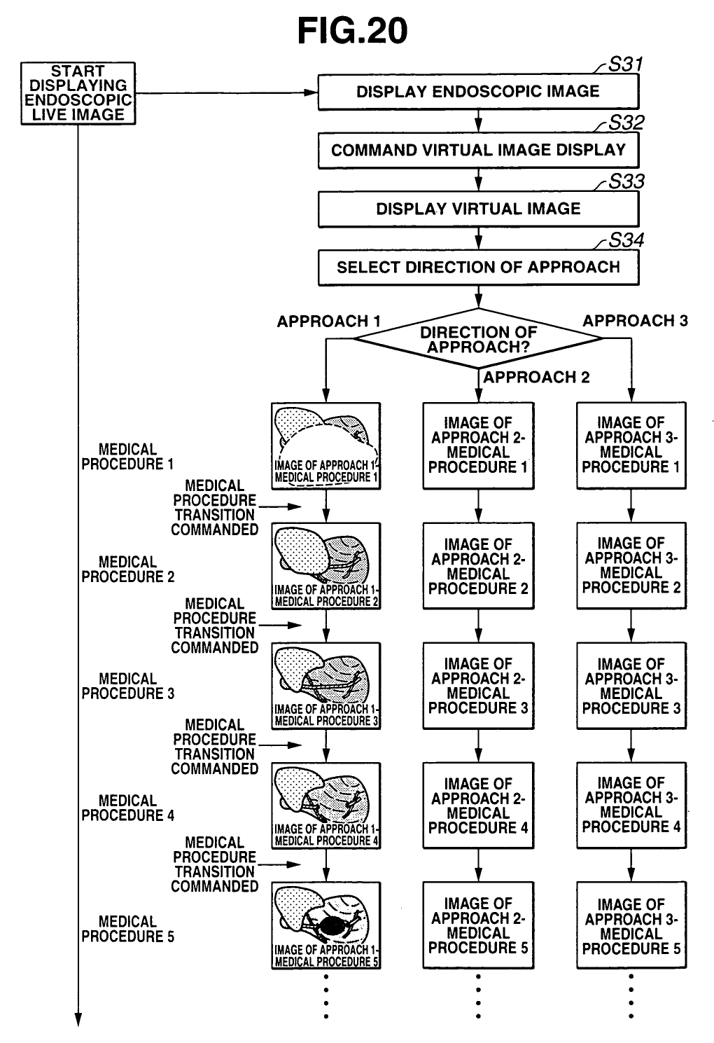

After the medical procedure virtual image in accordance with the progress of the medical procedure for each direction of approach is stored in the rendering image database 23b in the database unit 23 in this way, the medical procedure is started by the operator. Then, when the camera head 2d picks up an observation image of an internal part of a subject, an endoscopic image 200 is displayed on the endoscopic image monitor 13 in step S31, as shown in FIG. 20.

Then, if the operator produces a voice such as “Virtual display” in accordance with the progress of the medical procedure in step S32, the voice input microphone 12B, for example, detects the voice in step S33, and the system controller 10 recognizes the operator's command by voice recognition processing. Then, if the system controller 10 recognizes the operator's command, the system controller 10 commands the CPU 25 in the virtual image creating unit 11 to display the medical procedure 1 virtual image 110 (1) shown in FIG. 5 on the monitor 17a.

The medical procedure 1 virtual image 110(1) is selected with reference to the table of approach 1 by default.

Next, if the operator produces a voice such as “Approach 1” in step S34, the voice input microphone 12B, for example, detects the voice, and the system controller 10 recognizes the operator's command by voice recognition processing. Then, if the system controller 10 recognizes the operator's command, the system controller 10 commands the CPU 25 in the virtual image creating unit 11 to display the medical procedure i virtual image 110 (i) on the monitor 17a with reference to the table of approach i for the number, “i”, of the specified direction of approach.

Then, as in the first embodiment, the operator produces a voice such as “Virtual display” in accordance with the progress of the medical procedure to command the transition of medical procedure virtual images so that the medical procedure virtual images 110 can be sequentially displayed on the monitor 17a based on the operator's voice command and in accordance with the progress of the medical procedure.

In addition to the advantages of the first embodiment, the second embodiment can provide the virtual image suitable for medical procedure support in real time during the medical procedure operation even when the direction of approach to the affected part of the treatment device such as forceps is determined based on the position of the affected part in the focus organ in the beginning of the medical procedure under endoscopic observation.

The invention may be obviously embodied in widely different forms without departing from the spirit and scope of the invention and based on the invention. The invention is only defined by the appended claims rather than the specific embodiments.

1. A medical procedure support system, comprising:
an image reading unit for reading virtual image data from a storage unit that stores multiple pieces of the virtual image data relating to a subject, which correspond to steps of a medical procedure;
a specifying unit for specifying a step of the medical procedure; and
a control unit for controlling the image reading unit based on the step of the medical procedure specified by the specifying unit.

2. The medical procedure support system according to claim 1, further comprising:
a virtual image managing unit for managing the multiple pieces of virtual image data stored in the storage unit for each approach to a focus part of the subject.

3. The medical procedure support system according to claim 1, wherein the virtual image data is created from image data created by a CT apparatus that picks up an X-ray tomographic image.

4. The medical procedure support system according to claim 2, wherein the virtual image data is created from image data created by a CT apparatus that picks up an X-ray tomographic image.

5. The medical procedure support system according to claim 1, wherein the specifying unit specifies the information relating to a step of the medical procedure based on voice information from a voice input unit by which voice is inputted.

6. The medical procedure support system according to claim 2, wherein the specifying unit specifies the information relating to a step of the medical procedure based on voice information from a voice input unit by which voice is inputted.

7. The medical procedure support system according to claim 3, wherein the specifying unit specifies the information relating to a step of the medical procedure based on voice information from a voice input unit by which voice is inputted.

8. The medical procedure support system according to claim 4, wherein the specifying unit specifies the information relating to a step of the medical procedure based on voice information from a voice input unit by which voice is inputted.

9. A medical procedure support system, comprising:
an endoscope having an image pickup unit for picking up an internal part of a body cavity of a subject;
an endoscopic image creating unit for creating an endoscopic image from an image signal from the image pickup unit;
a storage unit for storing information relating to steps of a medical procedure and virtual image data relating to the subject, which are associated with each other;
an image reading unit for reading the virtual image data from the storage unit;
a specifying unit for specifying the information relating to a step of the medical procedure under the endoscopic image observation; and
a control unit for controlling the reading of virtual image data by the image reading unit based on the information relating to the step of the medical procedure specified by the specifying unit.

10. The medical procedure support system according to claim 9, further comprising:
a virtual image managing unit for managing multiple pieces of virtual image data stored in the storage unit for each approach to a focus part of the subject.

11. The medical procedure support system according to claim 9, wherein the virtual image data is created from image data created by a CT apparatus that picks up an X-ray tomographic image.

12. The medical procedure support system according to claim 10, wherein the virtual image data is created from image data created by a CT apparatus that picks up an X-ray tomographic image.

13. The medical procedure support system according to claim 9, wherein the specifying unit specifies the information relating to a step of the medical procedure based on voice information from a voice input unit by which voice is inputted.

14. The medical procedure support system according to claim 10, wherein the specifying unit specifies the information relating to a step of the medical procedure based on voice information from a voice input unit by which voice is inputted.

15. The medical procedure support system according to claim 11, wherein the specifying unit specifies the information relating to a step of the medical procedure based on voice information from a voice input unit by which voice is inputted.

16. The medical procedure support system according to claim 12, wherein the specifying unit specifies the information relating to a step of the medical procedure based on voice information from a voice input unit by which voice is inputted.

17. A medical procedure support method, comprising:
an image reading step of reading virtual image data from a storage unit that stores multiple pieces of virtual image data relating to a subject, which correspond to steps of a medical procedure;
a specifying step of specifying a step of the medical procedure; and
a control step of controlling the reading by the image reading step based on the step of the medical procedure specified by the specifying step.

18. The medical procedure support method according to claim 17, further comprising:
a virtual image managing step of managing the multiple pieces of virtual image data stored in the storage unit for each approach to a focus part of the subject.

19. A medical procedure support method, comprising:
an endoscopic image creating step of creating an endoscopic image from an image signal from an image pickup unit of an endoscope that picks up an internal part of a body cavity of a subject;
a storage step of storing information relating to steps of a medical procedure and virtual image data relating to the subject, which are associated with each other, in a storage unit;
an image reading step of reading the virtual image data from the storage unit;
a specifying step of specifying the information relating to a step of the medical procedure under the endoscopic image observation; and
a control step of controlling the reading of virtual image data by the image reading step based on the information relating to a step of the medical procedure specified by the specifying step.

20. The medical procedure support method according to claim 19, further comprising:
a virtual image managing step of managing the multiple pieces of virtual image data stored in the storage unit for each approach to a focus part of the subject.

21. A medical procedure support system, comprising:
image reading means that reads virtual image data from a storage unit that stores multiple pieces of the virtual image data relating to a subject, which correspond to steps of a medical procedure;
specifying means that specifies a step of the medical procedure; and
control means that controls the image reading means based on the step of the medical procedure specified by the specifying means.

22. The medical procedure support method according to claim 21, further comprising:
virtual image managing means that manages the multiple pieces of virtual image data stored in the storage unit for each approach to a focus part of the subject.

23. A medical procedure support system, comprising:
an endoscope having image pickup means that picks up an internal part of a body cavity of a subject;
endoscopic image creating unit that creates an endoscopic image from an image signal from the image pickup means;
storage means that stores information relating to steps of a medical procedure and virtual image data relating to the subject, which are associated with each other;
image reading means that reads the virtual image data from the storage means;
specifying means that specifies the information relating to a step of the medical procedure under the endoscopic image observation; and
control means that controls the reading of virtual image data by the image reading means based on the information relating to the step of the medical procedure specified by the specifying unit.

24. The medical procedure support system according to claim 23, further comprising:
virtual image managing means that manages the multiple pieces of virtual image data stored in the storage means for each approach to a focus part of the subject.
